(19) United States
(12) Patent Application Publication
(10) Pub. No.: US 2024/0315406 A1
(43) Pub. Date: Sep. 26, 2024

(54) MOBILE ELECTRONICS DEVICE

(71) Applicants: Alexander RECH, Graz (AT); Ines WÖCKL, Graz (AT)

(72) Inventors: Alexander RECH, Graz (AT); Ines WÖCKL, Graz (AT)

(73) Assignee: Flasher GmbH, Graz (AT)

(21) Appl. No.: 18/579,528

(22) PCT Filed: Jul. 15, 2022
(86) PCT No.: PCT/AT2022/060250

§ 371 Date: Jan. 16, 2024

(30) Foreign Application Priority Data
Jul. 16, 2021 (AT) A 127/2021

(51) Int. Cl. A44C 5/14; A44C 27/00; G04B 37/14

(52) U.S. Cl. CPC A44C 5/14; A44C 27/008; G04B 37/1486

(57) ABSTRACT

The present invention relates to a mobile electronics device (10) to be fastened to a human body part (200), comprising an electronics housing (20) having electronics components (30) situated therein, wherein a flexible fastening arm (40) is situated in each case on the electronics housing (20) on two oppositely oriented wall portions (22), which fastening arms extend away from the electronics housing (20) and are flexibly deformable between a fastening position (FP), fastened to the body part (200), and a release position (RP), releasing the body part (200), and wherein, in the release position (RP), the fastening arms (40) are preloaded towards the fastening position (FP) with a preloading force (PF).

The present invention relates to a mobile electronics device to be fastened to a human body part and an electronics set having at least two such mobile electronics devices.

It is known for mobile electronics devices to be provided with a means of fastening them to the human body when in use. These include for example so-called smartwatches, fitness trackers or the like. These are usually fastened to the arm with flexible wristbands in order to be available to the user for mobile use. These flexible wristbands can be placed around the respective body part manually, to be held in this fastening position with locking mechanisms. The disadvantage of these solutions is the relatively high effort required to wrap the flexible wristbands around the body part and fasten them in place.

Also known are snap springs which are used as passive wristbands. Such snap bands are long base bodies designed as snap springs which can be placed around a body part and snap into a fastening position. The disadvantage of these solutions is the high inherent tension in these snap springs, which makes a combination with electronic components difficult and sometimes even impossible. This applies in particular to the fact that the preloading of such snap springs makes it more difficult to seal the electronic components against water and dust and to protect these electronic components from mechanical stress. Another disadvantage of these known snap bands is that they are also complicated to use.

It is the object of the present invention to remedy, at least in part, the disadvantages described above. In particular, it is the object of the present invention to create, in a cost-effective and simple manner, a quick and in particular one-handed means of attaching a mobile electronics device to a body part. Preferably, a protected arrangement of the electronic components is ensured at the same time.

The above object is achieved by a mobile electronics device with the features of claim 1 and an electronics set with the features of claim 22. Further features and details of the invention are disclosed in the dependent claims, the description and the drawings. Naturally, features and details which are described in connection with the mobile electronics device according to the invention also apply in connection with the electronics set according to the invention and in each case vice versa, so that with regard to disclosure mutual reference is or can always be made to the individual aspects of the invention.

According to the invention, a mobile electronics device is designed to be fastened to a human body part. The mobile electronics device has an electronics housing with electronic components arranged therein. On this electronics housing, a flexible fastening arm is in each case arranged on two oppositely oriented wall portions. These fastening arms extend away from the electronics housing and are flexibly deformable between a fastening position, fastened to the body part, and a release position releasing the body part. In the release position, the fastening arms are also preloaded towards the fastening position with a preloading force.

A core idea of the invention is the provision of a mobile electronics device, as used for example in smartwatches, fitness trackers or the like. In particular, a core idea is a mobile signalling or communication unit which is able to output light signals from the body part, as will be explained later. For this purpose, appropriate electronic components in the form of illuminants, battery devices or the like can be provided.

The core idea of the invention of an easy and simple means of attachment to the body part of the user is ensured by the provision of two separate fastening arms. However, in contrast to the flexible wristbands of watches, these fastening arms are pre-tensioned. The preloading is present in the release position of the fastening arms and is directed towards the fastening position. This means that a release of this preloading force in the release position leads to a movement of the fastening arms into the fastening position guided by the preloading force. In a similar way to the snap springs, as explained in the introduction, this allows an automatic movement from the release position to the fastening position, effected by the preloading force, to take place, for example in a snapping manner. In order to apply and attach the device to a body part, the user can thus leave the fastening arms in the pre-tensioned release position and guide them into a desired position on the user's body part. As soon as this positioning in which the mobile electronics device is to be fastened is reached, the user releases the preloading force present in the release position, for example by means of corresponding pressure points on the fastening arms, so that the two fastening arms are moved through the preloading force from the release position into the fastening position, whereby, in particular, a residual preloading force remains in the fastening position. This residual preload force can also be understood as the remaining fastening force and serves to create a force-fitting connection between the fastening arms and the body part in the fastening position.

As can be seen from the above explanation, a one-handed fastening to the body part can now take place. The user grips the electronics housing with one hand and guides the two fastening arms, for example, onto two different sides of their upper arm. As soon as this position of the fastening arms has been reached, in particular when the underside of the electronics housing touches the outside of the upper arm as a body part, the user presses on the pressure points of the two fastening arms and thus triggers the preloading force secured in the release position. The preloading force triggered in this way moves the fastening arms into a curved fastening position around the upper arm, for example in a snapping movement, and thus secures the mobile electronics device in this fastening position.

In addition to the one-handed, very simple operation when fastening the device to the body part, this also allows a correspondingly one-handed operation when removing the device from the body part. Starting out from the explanation in the previous paragraph, the user can again grip the attached mobile electronics device by the electronics housing and simply pull it off, against the increasing preloading force, by redeforming the fastening arms from the fastening position. In particular, they will move the fastening arms completely into the release position through counterpressure against the body part, so that the preloading force is secured again by the fastening arms snapping into this release position. The preloaded release position is thus automatically preloaded again for a future fastening process after the mobile electronics device has been pulled off the body part.

In addition to the improved ease of use and in particular the possibilities of one-handed operation, the protection of the electronic components of a mobile electronics device can be guaranteed very simply. Due to the fact that a preloading force and a fastening force act on the fastening arms, these forces must be supported on the electronics housing. This takes place on the oppositely oriented wall portions, in particular in the mounting sections which will be explained later. A decisive advantage of an electronics device according to the invention is that the electronics housing itself, in particular the electronic components arranged therein, is not affected by the forces of the fastening arms. In particular, the electronic components are positioned in the electronics housing in a force-free manner or free from the preloading force and/or the fastening force of the fastening arms. This is possible because the two fastening arms are separated from each other and in particular do not extend through the electronics housing.

It can be seen from the above explanation that there is a functional separation between the force-loaded fastening functionality in the fastening arms and the force-free housing of the electronic components in the electronics housing. This makes it possible to protect the electronic components more effectively and/or more easily and thus increase the longevity of the functionality of the mobile electronics device. In addition, the desired sealing against dust or water can be provided much more easily, since the corresponding regions of the electronics housing which are to be sealed are not subjected to the fastening forces and the preloading forces. In other words, a simplified operation of a mobile electronics device for the user can now be combined with an increased durability in terms of freedom from forces acting on the electronic component and impermeability to dust and water. This force-free design allows cost-effective production.

It should also be pointed out that the electronic components can of course take a wide variety of forms. For example, any kind of sensors, such as gyroscopes, force sensors, light sensors or the like, are conceivable. Means of communication, for example Bluetooth elements, Bluetooth low energy elements, UWB (Ultra Wide Band) elements, NFC elements or mobile communication elements can also be selected as electronic components. Vibration motors, acoustic actuators, the LEDs or battery devices already explained, as well as control modules for the control of the other electronic components, can also be arranged as electronic components in the electronics housing.

It can be advantageous if, in a mobile electronics device according to the invention, in the release position the two fastening arms have a preloading deformation which holds the preloading force in the release position. This preloading deformation serves in particular to secure or engage the fastening arms in the release position so that, for example by activating pressure points on the fastening arms, this preloading deformation can be released in order to release the preloading force to bring about a movement of the fastening arm from the release position to the fastening position. In the reverse direction, the fastening arm will in the release position assume the preloading deformation and thus secure the preloading force. For example, the fastening arms can be designed as separate snap springs which have an additional preloading curvature, in particular transversely to the fastening curvature, as will be explained later. Depending on the nature and formation of the preloading deformation, the positioning of the pressure points for releasing this preloading deformation can also be shifted. Thus, it is possible to predefine, in the design of the fastening arms, at which points the pressure points for the fastening operation are to be positioned.

It is advantageous if, in a mobile electronics device according to the previous paragraph, the preloading deformation has a preloading curvature the orientation of which orientation differs from a fastening curvature in the fastening position. In particular, a mounting section of the electronics housing is provided which predetermines this preloading curvature. A preloading deformation in a preloading curvature is a particularly simple form of such a preloading deformation. The corresponding radii of curvature and thus the underlying circles of curvature of the preloading curvature and the fastening curvature are preferably oriented transversely, in particular perpendicular to each other. The use of a mounting section in the electronics housing to clamp the fastening arms can predetermine this preloading curvature through a corresponding curved mounting surface. This makes it possible to support this preloading curvature in the mounting section in a force-free manner and keep the rest of the electronics housing force-free even with high preloading forces. The more pronounced the preloading curvature, the easier it is for the fastening arm to snap from the release position to the fastening position on releasing the preloading deformation. The pressure point for releasing the preloading force also shifts in the direction of the electronics housing, further improving one-handed operation. In particular, it is then possible to press both pressure points of the two fastening arms at the same time using a pincer grip, so that the two fastening arms are snapped into the fastening position at substantially the same time.

In addition, it is advantageous if, in a mobile electronics device according to the invention, the electronics housing has a mounting section on each of the oppositely oriented wall portions, in which a complementary mounting section of the respective fastening arm is received. As explained in the preceding paragraph, such a mounting section may have a curved surface in order in particular to predetermine the preloading curvature. Of course, complementary, preferably likewise curved mounting surfaces on the upper side and underside of this mounting section are also conceivable in order to allow the fastening arm to be clamped on both side surfaces. The fastening of the fastening arm in the mounting section can be achieved by clamping, screwing, overmoulding, snap-locking or similar means. Compared to the total length of the fastening arms, the clamping length in the mounting section is preferably relatively short. For example, the clamping length or reception length of the fastening arms can be in the range of about 1% to about 5% of the total length of the fastening arms.

Further advantages can be achieved if, in a mobile electronics device according to the invention, the two fastening arms are of the same length or substantially the same length. This means that, in particular, an identical or substantially identical formation of the two fastening arms is achieved. On the one hand, such an identical design of the fastening arms has the advantage that the mobile electronics device can be attached to the body part of the user in an identical manner in both possible orientations. On the other hand, designing both fastening arms as identical parts is advantageous in that the complexity of the mobile electronics device is reduced through the number of identical components. In addition to easier and simpler assembly, a reduction in component costs can also be achieved in this way. It should also be noted that the length of the fastening arms is preferably adapted to the diameter of the body part to which the device is usually intended to be attached. For example, if the body part is the user's upper arm, a minimum body part circumference and a maximum body part circumference can be defined depending on the person. The fastening arms are designed in such a way that they still ensure a sufficient wrapping-around to achieve a firm and secure fastening on the body part at the maximum circumference of the body part. For the minimum circumference of the body part, the fastening arms are advantageously short enough that an overlap of the fastening arms in the fastening position does not exceed one third of the total length of the respective fastening arm.

Furthermore, it can be advantageous if, in a mobile electronics device according to the invention, at least one of the two flexible fastening arms has a pressure point, wherein the action of a compressive force on this pressure point moves the fastening arm into an “loosen” position in which the preloading force is released and the fastening arm is moved by the released preloading force into the fastening position. Thus, the “loosen” position represents a defined intermediate position between the release position and the fastening position. In other words, the “loosen” position is the boundary between two ranges which distinguish a movement in the opposite direction. If the fastening arm is in the range between the release position and the “loosen” position, the fastening arm is stable in the release position. This can for example be achieved by a restoring force acting in the direction of the release position. As soon as such a restoring force is overcome by applying the compressive force via at least one pressure point, the fastening arm moves in the direction of the “loosen” position. When the “loosen” position is exceeded, the fastening arm assumes a shape which releases the preloading force. For example, in the “loosen” position, a preloading curvature is at least partially released, so that from this “loosen” position the fastening arm is automatically moved to the at least one fastening position without further application of force via the pressure point. This movement ends as soon as a resistance is encountered, for example through a surface of the body part to which the electronics device is to be attached. In other words, the fastening arm snaps into the fastening position or in the direction of the fastening position after exceeding the “loosen” position.

It is also advantageous if, in a mobile electronics device according to the invention, both fastening arms have a pressure point, in particular at an identical or substantially identical position. Preferably, the two pressure points are distributed symmetrically on the two fastening arms with respect to the electronics housing. One advantage that can be achieved in this way is that the two fastening arms can be of identical design and, when mounted symmetrically, form the fastening function on both sides. This reduces complexity due to a reduced number of components and in this way the costs are minimised. This also makes it possible to operate the two pressure points manually with one hand in the form of a pincer grip and in this way carry out the fastening operation. In addition, the symmetry makes it possible to apply the device to the body part in two different orientations, which further improves intuitive usability.

It is also advantageous if, in a mobile electronics device according to the invention, at least one pressure point has a gripping distance from the electronics housing that is less than a gripping width of a human hand. In this way, the pincer grip of a human hand, which has already been described in the previous paragraph, becomes even easier to use. In particular, the entire electronics device can be held at the pressure points with such a pincer grip. Once the device has been placed on or around the corresponding body part, the user can increase the pressure by means of the pincer grip until the applied pressure force exceeds a restoring force of the fastening arms. This initiates the movement into the “loosen” position, as a result of which the snapping movement of the fastening arms then completes the fastening operation. This clearly demonstrates the ergonomic and, above all, one-handed operability which for example allows one-handed application to the user's opposite upper arm.

It also has advantages if, in a mobile electronics device according to the invention, at least one pressure point is arranged centrally or substantially between edges of the fastening arm. Particularly in embodiments with a preloading curvature, the pressure point can be arranged in a lower-lying region between the edges of the fastening arm. In this way, the upwardly curved edges form an anti-slip safeguard and at the same time guide the user's fingers to the correct pressure point. This too further improves security and ease of use.

It is also advantageous if, in a mobile electronics device according to the invention, at least one pressure point has a pressure point surface, the surface normal of which is oriented perpendicular or substantially perpendicular to a main extension of the fastening arm in the preloading position. The surface normal is a vertical line running through the pressure point surface and can in particular also be referred to as the perpendicular bisector of the pressure point surface. In other words, in this embodiment the compressive force is applied perpendicular or substantially perpendicular to the pressure point surface and thus also to the main extension of the fastening arms. This facilitates the precisely-directed application of force and reduces the risk of slipping out of this position.

In addition, it has advantages if, in a mobile electronics device according to the invention, at least one of the fastening arms has a counter-pressure point at a distance from the pressure point, wherein an action of a counter-compressive force directed opposite to the compressive force moves the fastening arm from the “loosen” position to the release position and secures the preloading force. The counter-pressure point is preferably more distant from the electronics housing than the pressure point of the respective fastening arm. Also, counter-pressure points are preferably arranged on both fastening arms, in particular symmetrically to the electronics housing. The counter-pressure points allow a precise guidance of the fastening arms into the release position and the securing of the preloading force. In particular, the application of the counter-compressive force supports a deformation of the respective fastening arm to assume a preloading deformation, for example in the form of a preloading curvature.

It is also advantageous if, in a mobile electronics device according to the invention, at least one of the two fastening arms has a light conductor, in particular in the form of a light conducting layer, wherein, in the electronics housing, at least one illuminant is arranged as an electronic component in light-transmitting contact with the light conductor. This provides an electronic component in the form of one or more illuminants, which allows external communication by means of light. A significant advantage of this design is that the illuminants are integrated into the electronics housing as electronic components, and the fastening arms can thus be designed without illuminants and thus also without electronic components. For example, the illuminants, for example in the form of LEDs, can be mounted on a circuit board in the electronics housing so as to be force-free, dustproof and waterproof. By means of a surface contacting with a coupling surface of the light conductor, it is possible to introduce emitted light from the illuminants into the light conductor and to guide it along its extension along the fastening arms. The light can be decoupled at separate components or reflective surfaces of the fastening arm or at the decoupling surfaces of the light conductor itself, which will be explained later. This makes it possible to provide a signalling function with the electronic component housed completely within the electronics component and an emission surface, and thus a signalling surface, which is integrated into the fastening arms. This combination is only possible in such a simple manner because the fastening arms are designed separately from the electronics housing in terms of their fastening and preloading forces, so that mechanical loads on the electronic components in the form of the illuminants can substantially be completely avoided here. The design of the light conductor in the form of a light conducting layer is particularly advantageous, since it can in particular cover the surface of the fastening arm completely or substantially completely. This light conducting layer can be attached to a base body of the respective fastening arm, or it can be held in the desired position on the fastening arms through combination with a protective layer, which will be explained later. Of course, more than one illuminant, in particular a plurality of identical illuminants in the form of LEDs, can also be arranged within the electronics housing. The light conductor itself has at least the same flexibility as is also provided for the fastening arms to allow the deformation between the release position and the fastening position. Preferably, in this embodiment the illuminants are all identical and can in particular provide several colours at the same time. A colour change is also preferably possible with such LEDs. A mechanical fastening of the light conductor to the base body via depressions or openings in the base body is also conceivable.

Further advantages can be achieved if, in a mobile electronics device, the light conductor has decoupling surfaces to allow the conducted light to be decoupled in a decoupling direction away from the fastening arms. Such decoupling surfaces can for example be slits or point-like depressions in the light conductor, in particular the light conducting layer. These are advantageously attached to the underside of the light conductor, i.e., on the side of the light conductor that contacts the upper side of the fastening arms. The fastening arms are preferably formed by a solid base body, in particular of metal. The decoupling surfaces can be simple indentations or slits in the light conductor. Alternatively or in addition to the slots, printed dots or other surface shapes are also conceivable as decoupling surfaces on a side of the base body opposite the decoupling direction. However, additional reflection surfaces or separating elements can also be provided in order to form individual light conducting channels within the light conductor. The decoupling surfaces can form geometric shapes which can be perceived from outside the mobile electronics device as light elements or light forms on being irradiated with light beams by the illuminants. These forms for the decoupling surfaces are also preferably point-formed and/or linear since, for a signalling function, these are more readily and clearly perceptible by other people than a two-dimensional radiation of light.

It can also be advantageous if, in a mobile electronics device according to the invention, the decoupling surfaces increase in size with increasing distance from the at least one illuminant. Since a part of the amount of light is always decoupled and emitted at each decoupling surface over the length of the fastening arm, the available amount of light decreases with increasing distance away from the illuminants. In order to be able to provide an advantageous adjustment of the luminosity or a deliberate distribution of different luminosities over the entire course of the light conductor, at the beginning, i.e., close to the illuminants, a small decoupling surface will emit a small percentage of a large amount of light, while at a more distant point from the illuminants, a larger decoupling surface will emit a larger percentage of light from the total amount of light, which, however, is already reduced at this point. To achieve other distributions of luminosity, correspondingly different surface area relationships must be used accordingly. Preferably, the increase in the decoupling surface correlates with the decrease in the amount of light, so that a uniform emission of light from the decoupling surfaces is achieved.

It is also advantageous if, in a mobile electronics device according to the invention, the fastening arms have a base body which is surrounded by a protective layer. Such a protective layer can preferably completely surround the fastening arm and in particular also extend into the mounting section of the electronics housing. This extension can be designed in the form of sealing lips in order to be able to provide, here too, a design that is as dustproof and watertight as possible. The protective layer is also adapted to the necessary flexibility to allow moveability with reversible deformation between the fastening position and the release position. In order to provide this function the protective layer can for example comprise a silicone material. Also, the protective layer can be designed as protective socks, in particular as silicone socks, and in this way the base body can be inserted into the interior of such a protective sock. This insertion can be facilitated if the silicone sock is held in an open position, for example by means of negative pressure. It is also conceivable to form the protective layer for the base body by complete overmoulding. When selecting the material of the protective layer, an increase in the amount of light emitted can be achieved by selecting advantageous refractive indices. A further improvement of the optical properties can be achieved by forming an air layer between the light conductor and the protective layer. A light conductor can be formed integrally with this protective layer or can be separately incorporated therein. In addition, or alternatively, colour layers are also conceivable, which are either integrated into the protective layer or applied to a base body of the fastening arms. Such colour layers, in particular foil, powder or varnish layers, are able to provide a personalisation of the mobile electronics device through appropriate colour selection and/or improved reflective properties, in particular through the use of passive reflective elements.

It is also advantageous if, in a mobile electronics device according to the invention, the fastening arms are free or substantially free of electronic components. This allows the fastening arms to concentrate completely on the fastening function and thus the correspondingly necessary preloading forces and fastening forces can be applied independently of any otherwise necessary protection of electronic components. The flexibility and also the movements of the fastening arms serve the fastening function without leading to wear on the electronic components performing the electronics function.

Further advantages can be achieved if, in a mobile electronics device according to the invention, housing light sources are arranged in the electronics housing to emit light through housing windows. These are in particular separate from the already explained light sources intended to couple light into the light conductor of the fastening arms. The housing light sources are preferably likewise arranged on a circuit board. Housing windows can be made of the same material as the rest of the housing sections of the electronics housing. However, they are preferably made transparent by an appropriate choice of materials or by polishing the surface, so that the highest possible proportion of the light generated by the housing light sources also reaches the outside through these housing windows and can be perceived by persons there.

It brings further advantages if, in a mobile electronics device according to the invention, the electronic components are at least partially arranged on a common circuit board, wherein, in particular, fastening means attach the circuit board to the electronics housing, within the electronics housing. A circuit board can be pre-produced and standardised, so that the final assembly of these electronic components arranged on the circuit board within the electronics housing can be carried out very easily. The use of fastening means allows the circuit board to be attached to the electronics housing, for example by means of evenly distributed screws. In this way, it is also possible to apply the described illuminants in the form of the LEDs to the fastening arms, specifically to the light conductor, preferably in a gap-free manner. Other electronic components, for example vibration motors, can also be correlated specifically and exactly with the electronics housing in this way, so that, in the case of a vibration motor, its vibration can be transmitted to the underside of the electronics housing. Such a circuit board can be made of a wide variety of materials. To achieve significantly better heat dissipation, in particular when energy-intensive and heat-generating continuous operation is to take place, the circuit board can be made of an aluminium material.

It also has advantages if, in a mobile electronics device according to the invention, fixing means are arranged on the housing to fix the fastening arms to the electronics housing, these having alignment domes for aligning the fastening arms to the electronics housing. Such fixing means can be used for reversible or irreversible fixation. Thus, in principle, clamping, snapping or snap-locking, but also the use of a screw connection for such fixing means is conceivable within the context of the present invention. Preferably, an odd number of fixing means is provided for each fastening arm in order to ensure the most exact positioning and also application of the already explained preloading force. The alignment domes can, for example, protrude through corresponding mounting openings in the circuit board and/or the fastening arms to ensure the positioning of the individual components relative to each other during assembly.

It is also advantageous if, in a mobile electronics device according to the invention, the electronics housing has an underside curvature on its underside allowing it to lie at least partially flat against the body part. This underside curvature is preferably adapted to a fastening curvature in the fastening position, and continues this. The underside curvature serves to form an average value for body parts with different body part diameters, so that it can lie at least partially flat against as many different body parts as possible. In addition to increased comfort when wearing the attached mobile electronics device on the body part, a flat contact also serves to improve transmission, for example, if vibrations are to be generated by a vibration motor as an electronic component.

It can also be advantageous if, in a mobile electronics device according to the invention, the arm ends of the fastening arms arranged at a distance from the electronics housing have a rounded edge. In addition to improved handling, this leads to a reduced risk of injury compared with the otherwise possibly sharp-edged design. A reduction in the weight of the fastening arms can also be achieved with the same functionality.

A further subject matter of the present invention is an electronics set having at least two mobile electronics devices according to the invention. Thus, an electronics set according to the invention has the same advantages as have been explained in detail with reference to a mobile electronics device according to the invention. If the mobile electronics devices have a defined orientation for a fastening position on a left- and a right-hand body part of the user, the electronics set is preferably designed with one of these design variants. A retail set for use on both sides of the body with corresponding body parts can thus be offered. In order to reduce the space taken up by the electronics set in a packaging unit, the fastening arms are preferably arranged in the fastening position and wrapped around each other.

Further advantages, features and details of the invention are disclosed in the following description, in which exemplary embodiments of the invention are described in detail with reference to the drawings. The features mentioned in the claims and in the description may in each case be essential to the invention individually or in any combination. In each case schematically:

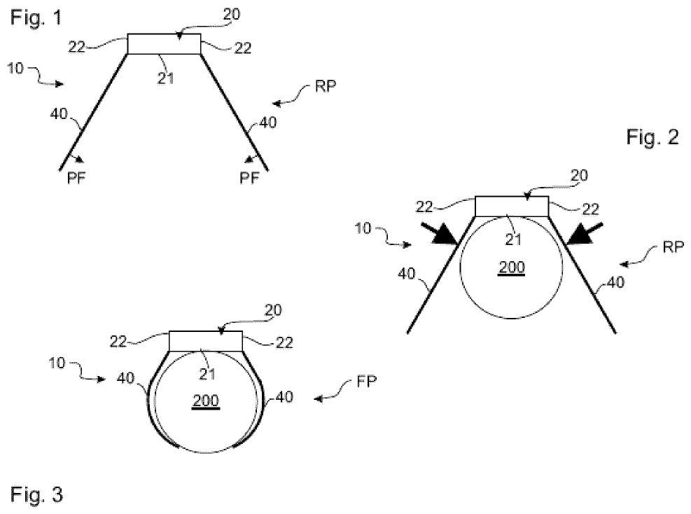

FIG. 1 shows an embodiment of a mobile electronics device in the release position,

FIG. 2 shows the embodiment of FIG. 1 during the fastening operation,

FIG. 3 shows the embodiment of FIGS. 1 and 2 in the fastening position,

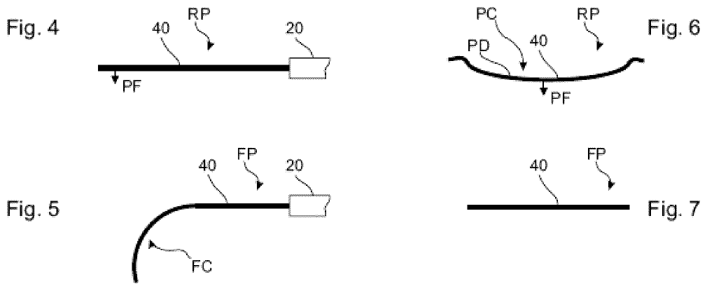

FIG. 4 shows a partial representation of another embodiment of a mobile electronics device,

FIG. 5 shows the embodiment of FIG. 4 in the fastening position,

FIG. 6 shows a cross-sectional representation of the embodiment of FIG. 4,

FIG. 7 shows a cross-sectional representation of the embodiment of FIG. 5,

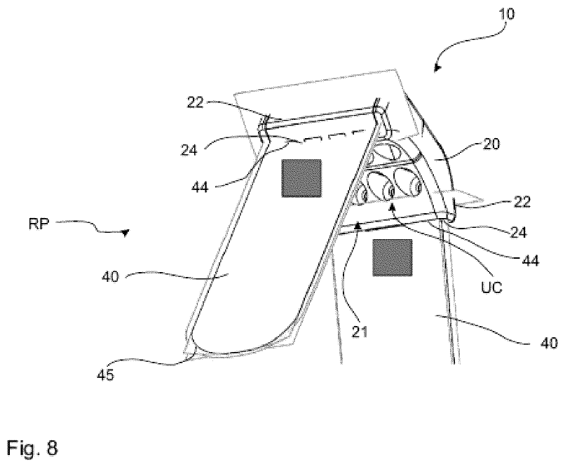

FIG. 8 shows a further embodiment of a mobile electronics device according to the invention,

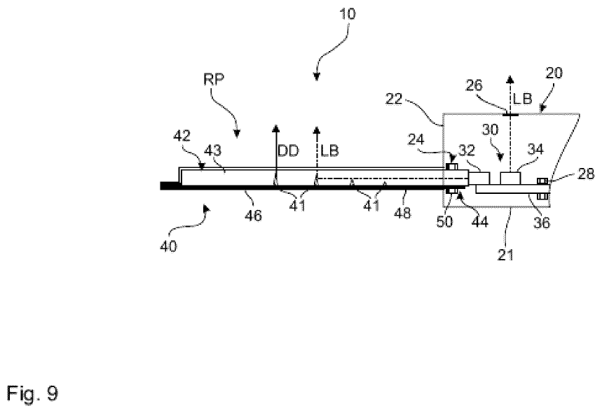

FIG. 9 shows a partial cross-section through another embodiment of a mobile electronics device according to the invention,

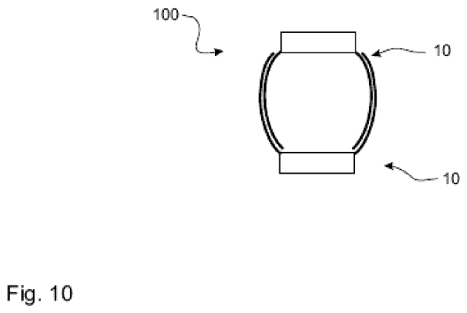

FIG. 10 shows an embodiment of an electronics set according to the invention.

FIGS. 1 to 3 show a particularly simple embodiment of a mobile electronics device 10 as well as the individual steps for fastening same to a body part 200. The body part 200 in this example can be an upper arm of a user.

To start the fastening process, the user has a mobile electronics device 10 according to FIG. 1. A number of electronic components 30, which are not shown in more detail here, are arranged in an electronics housing 20. Two wall portions 22 on the left and right sides of the electronics housing 20 each transition into a fastening arm 40, wherein the two fastening arms 40 extend downwards in a trapezoidal shape. The underside 21 of the electronics housing 20 is designed to be laid against the body part 200, as shown in FIG. 2.

In FIG. 1 it can also be seen that, in the shown release position RP of the fastening arms 40, each of the two fastening arms 40 applies a preloading force PF inwards in the direction of the fastening position FP according to FIG. 3. Due to a preloading deformation PD, which will be explained later, this preloading force PF is secured in the release position RP, so that this preloading force PF cannot be released without activity by the user.

If they wish to fasten it to the body part 200, the user grasps the mobile electronics device 10 of FIG. 1, for example by the two wall portions 22 of the electronics housing 20, and guides the two fastening arms 40 past the body part 200 on the left and right into the position shown in FIG. 2. In particular, the underside 21 of the electronics housing 20 is placed against the upper side of the body part 200. As the two thick arrows in FIG. 2 show, the user can now press pressure points here and in this way actively move the two fastening arms 40 out of the release position RP. This movement leads to the release of a preloading deformation PD, as will be explained later, and the fastening arms 40 are moved into the fastening position FP by the now released preloading forces PF. In other words, driven by the released preloading forces PF, the two fastening arms 40 now snap into the fastening position FP shown in FIG. 3. This shows the fastening position FP, wherein the two now curved fastening arms 40 partially wrap around the body part 200 and in this way fasten the electronics housing 20 to the body part 200 and secure it in this fastening position FP.

With reference to FIGS. 4, 5, 6 and 7, it will be explained once again in more detail how the preloading force PF can be secured. This refers in particular to an embodiment as explained with reference to FIGS. 1 to 3. FIG. 4 shows the still extended form of the fastening arms 40 in the release position RP, in which the preloading force PF is present in a secured manner. This securing is achieved by a preloading deformation PD, as shown in FIG. 6 with a view from the direction of the left-hand side of FIG. 4. This preloading deformation PD has a preloading curvature PC which prevents the fastening arm 40 from being released from the release position RP which is also shown here. This means that, while a preloading force PF is present, it is supported by the preloading deformation PD and is therefore not released.

If the preloading deformation PD is altered, e.g. by pressing in with a thumb or an index finger, in particular if the preloading curvature PC is released, the result is that the preloading force PF is no longer secured. Rather, this preloading force PF is released and thus by acting on the fastening arm 40 moves it into the fastening position FP shown in FIG. 5, with a fastening curvature FC. FIG. 7 shows a cross-section through the curved part of the fastening arm 40 of FIG. 5 along the longitudinal extension of this fastening arm 40. It can clearly be seen here that there is no longer any preloading curvature PC in this curved section of the fastening arm 40, so that the fastening position FP could be assumed accordingly. Depending on the embodiment, a small residual curvature is also possible here.

FIG. 8 shows another embodiment of a mobile electronics device 10 in a view from below. This is based on the design of the embodiment shown in FIGS. 1 to 7. In addition, however, the underside 21 of the electronics housing 20 is equipped here with an underside curvature UC, which is provided in order to allow the flattest possible contact with the outside of the body part 200. In particular, it continues the fastening curvature FC, at least in part, in order also to increase comfort when the device is worn on the body part 200. Two squares are used here to represent pressure sections situated close to the wall portions 22 which can be gripped and pressed with the user's thumb and index finger in a pincer grip in order to release the preloading deformation, as explained with reference to FIGS. 4 to 7. The representation in FIG. 8 shows the fastening arms 40 in the release position RP.

It can also clearly be seen in FIG. 8 that the upper ends of the fastening arm 40 each have a complementary mounting section 44 which is inserted into a mounting section 24 of the electronics housing 20. This insertion takes place in a curved manner, so that the contact surfaces present there predefine the preloading deformation PD for the fastening arm 40, in particular in a complementary manner. FIG. 8 also shows a variant in which the arm end 45 of the fastening arm 40 is rounded in order to reduce the risk of injury and minimise the weight of the fastening arms 40.

FIG. 9 shows a cross-section of a possible interior of a mobile electronics device 10. Here, the fastening arm 40 is, among other things, surrounded by a protective layer 48, which is provided as a sock-like silicone protection. This protective layer 48 also extends into a mounting section 44 of the electronics housing 20, where it serves as a dustproof and waterproof seal. In addition to a metallic base body 46, a light conductor 42 is arranged within the protective layer 48 as a light conducting layer 43. This light conducting layer 43 is transparent and can absorb light from an illuminant 32.

The illuminant 32 is arranged on a circuit board 36 within the electronics housing 20. This circuit board 36 is fastened to the electronics housing 20 with the help of fastening means 28, in this case a screw connection. In particular, this screw connection also serves to ensure a gap-free contact with the light conductor 42, so that light beams LB can be coupled from the illuminant 32 into the light conductor 42. This takes place in the region of the complementary mounting section 44, which allows a fixing arrangement of the fastening arm 40 in this position with the help of fixing means 50.

Slits, shown here in cross-section, are provided on the underside of the light conductor 42 as decoupling surfaces 41 allowing the decoupling of light beams LB. The decoupling direction DD is oriented substantially perpendicular to the base body 46, so that the light beams LB are decoupled in the same direction and are perceptible from the outside.

In addition, FIG. 9 also shows a variant in which a housing light source 34 is arranged on the circuit board 36. This can also consist of LEDs, which emit a light beam LB which is perceptible from the outside through a housing window 26. In this way, supplementary or, alternatively, additional light functions can be created as a signalling function.

FIG. 10 shows an electronics set 100 which comprises two mobile electronics devices 10. In particular, these can be electronics devices 10 according to FIGS. 1 to 3. If a distinction is made between the left and right sides of a user, this electronics set 100 consists of a left-hand and a right-hand embodiment of the mobile electronics device 10. It can also clearly be seen from this illustration that both fastening arms 40 of both electronics devices 10 are in the fastening position FP and wrap around each other, so that the space required for a packaging unit for this electronics set is reduced.

The above explanation of the embodiments only describes the present invention in the context of examples. Of course, individual features of the embodiments can, if technically expedient, be freely combined with each other without departing from the scope of the present invention.

### LIST OF REFERENCE SIGNS

10 mobile electronics device
20 electronics housing
22 wall portion
21 underside
24 mounting section
26 housing window
28 fastening means
30 electronic component
32 illuminant
34 housing light source
36 circuit board
40 fastening arm
41 decoupling surface
42 light conductor
43 light conducting layer
44 complementary mounting section
45 arm end
46 base body
48 protective layer
50 fixing means
100 electronics set
200 body part
FP fastening position
FC fastening curvature
RP release position
PF preloading force
PD preloading deformation
PC preloading curvature
UC underside curvature
DD decoupling direction
LB light beam

1. Mobile electronics device to be fastened to a human body part, comprising an electronics housing having electronics components situated therein, wherein a flexible fastening arm is arranged in each case on two oppositely oriented wall portions on the electronics housing, which extend away from the electronics housing and are flexibly deformable between a fastening position (FP), fastened to the body part, and a release position (RP), releasing the body part, and wherein, in the release position (RP), the fastening arms are preloaded towards the fastening position with a preloading force (PF).

2. Mobile electronics device according to claim 1, wherein in the release position (FP), the two fastening arms have a preloading deformation (PD) which holds the preloading force (PF) in the release position (PF).

3. Mobile electronics device according to claim 2, wherein the preloading deformation (PD) has a preloading curvature (PC) the orientation of which differs from a fastening curvature (FC) in the fastening position (FP), wherein in particular a mounting section of the electronics housing predefines this preloading curvature (PC).

4. Mobile electronics device according to claim 1, wherein the electronics housing in each case has a mounting section on the oppositely oriented wall portions in which a complementary mounting section of the respective fastening arm is received.

5. Mobile electronics device according to claim 1, wherein the two fastening arms are of the same length or substantially the same length.

6. Mobile electronics device according to claim 1, wherein at least one of the two flexible fastening arms has a pressure point, wherein the action of a compressive force on this pressure point moves the (PF) fastening arm into a “loosen” position in which the preloading force is released and the fastening arm is moved by the released preloading force (PF) into the fastening position (FP).

7. Mobile electronics device according to claim 6, wherein both fastening arms have a pressure point, in particular at an identical or substantially identical position.

8. Mobile electronics device according to claim 6, wherein the at least one pressure point has a gripping distance from the electronics housing which is less than a gripping width of a human hand.

9. Mobile electronics device according to claim 6, wherein the at least one pressure point is arranged centrally or substantially centrally between edges of the fastening arm.

10. Mobile electronics device according to claim 6, wherein the at least one pressure point has a pressure point surface the surface normal of which is oriented perpendicular or substantially perpendicular to a main extension of the fastening arm in the release position (FP).

11. Mobile electronics device according to claim 6, wherein at least one of the fastening arms has a counter-pressure point at a distance from the pressure point, wherein an action of a counter-compressive force, which opposes the compressive force, moves the fastening arm from the “loosen” position to the release position (RP) and secures the preloading force (PF).

12. Mobile electronics device according to claim 1, wherein at least one of the two fastening arms has a light conductor, in particular in the form of a light conducting layer, wherein, in the electronics housing, at least one illuminant is arranged as an electronic component in light-transmitting contact with the light conductor.

13. Mobile electronics device according to claim 12, wherein the light conductor has decoupling surfaces to allow the conducted light to be decoupled away from the fastening arms in a decoupling direction (DD).

14. Mobile electronics device according to claim 13, wherein the decoupling surfaces change, in particular increase in size, with increasing distance from the at least one illuminant.

15. Mobile electronics device according to claim 1, wherein the fastening arms have a base body which is surrounded by a protective layer.

16. Mobile electronics device according to claim 1, wherein the fastening arms are free or substantially free of electronic components.

17. Mobile electronics device according to claim 1, wherein housing light sources are arranged in the electronics housing to emit light through housing windows.

18. Mobile electronics device according to claim 1, wherein the electronic components are at least partially arranged on a common circuit board, wherein in particular fastening means fasten the circuit board to the electronics housing, within same.

19. Mobile electronic device according to claim 1, wherein fixing means are arranged on the electronics housing to fix the mounting arms to the electronics housing, these having in particular alignment domes for aligning the fastening arms to the electronics housing.

20. Mobile electronics device according to claim 1, wherein the electronics housing has an underside curvature (UC) on its underside allowing it to lie at least partially flat against the body part.

21. Mobile electronics device according to claim 1, wherein the arm ends of the fastening arms arranged at a distance from the electronics housing have a rounded edge.

22. Electronics set having at least two mobile electronics devices with the features of claim 1.
